(19) United States
(12) Patent Application Publication
(10) Pub. No.: US 2011/0135703 A1
(43) Pub. Date: Jun. 9, 2011

(54) Antimicrobial Coating for Surgical Implants and Method of Use

(76) Inventor: John I. Shipp, Atlantic Beach, FL (US)

(21) Appl. No.: 12/633,364

(22) Filed: Dec. 8, 2009

(51) Int. Cl. A61F 2/00; A61K 38/46; A61K 31/09; A61P 31/00

(52) U.S. Cl. 424/423; 424/94.67; 514/717

(57) ABSTRACT

A method of treating a surgical incision uses a material coated in an antimicrobial material. The material may be a synthetic mesh, an allograft, or a xenograft. The antimicrobial material is either tryclosan or lysostaphin. The coating of the material is performed either by adsorption or by covalent bonding.

### BACKGROUND

1. Field of the Invention

The present invention relates generally to an antimicrobial coating for surgical implants, in particular, to antimicrobial coatings for meshes used in hernia and soft tissue repair.

2. Description of the Related Art

It will be appreciated by those skilled in the art that the use of mesh for strengthening hernia and other soft tissue repair such as breast and pelvic floor reconstruction is well known. Synthetic and biological meshes have been implanted for this purpose. Synthetic meshes, generally, cause a high inflammatory response and as a result become ineffective and are encapsulated owing to the immune system's foreign body response, often requiring explantation. Decellurized biological materials cause less inflammatory response but are often weaker mechanically. Synthetic materials have no antimicrobial effects to combat infections, a common problem following implant. W. L. Gore produces a product known as DUALMESH® PLUS. The mesh material, expanded PTFE, is coated with two antimicrobial preservative agents, silver carbonate, and chlorhexidine diacetate, which act to inhibit bacterial colonization. The FDA classifies these inorganic compounds as toxic materials. Many patients have developed significant fever conditions following implant, consistent with toxic poisoning.

It is believed by many in the art that biological materials provide some antimicrobial effect. As the biological materials degrade, growth factors and peptides are released. The belief is that these elements possess antimicrobial properties that help ward off potential pathogens. Experience by others has shown that any such antimicrobial properties are inadequate to substantially reduce postoperative infection rates as compared to synthetics.

Approximately eighty percent of post hernia repair infections are caused by Staphylococcus aureus, a gram positive pathogen. Other pathogens, both gram positive and gram negative, have, to a lesser extent, been cultured from hernia infections.

Antimicrobial substances have been impregnated in implant devices such as central venous catheters and other transdermal devices. Bayston, in U.S. Patent Application 2007/0224243, discloses such impregnated devices and methods of making them. These devices are inserted through relatively small percutaneous access points, and infection sources can continually enter the body from the hospital environment. To be effective an antimicrobial agent associated with this type implant must last as long as the implant is in place and often must contain multiple agents that are effective against mutations. Because of the size of the opening, the pathogen loads are small (as compared to large surgical openings like open hernia repair wound sites), but continuous. Bayston discloses an impregnation method that slowly leaches out a mixture of antimicrobial agents that is effective up to 180 days. This impregnation method is ineffective for hernia procedures, however, partially because of the difference in the implant material and partially because of the magnitude of the bacterial challenge. Li, in U.S. Pat. No. 6,299,651, discloses the use of an antibacterial effusing textile fabric used to make clothing and other ware such as napkins. The process described therein produces an antimicrobial effect after at least 25 washing cycles, to counter low challenges of microbes that might be expected to be encountered by the user of the fabric.

Hernia repair is most often preformed in an open surgical procedure. Ventral hernia repair almost always involves large abdominal openings that subject the patient to potentially large one time challenges of pathogens. Once the abdominal cavity is closed following the repair, the potentially large pathogen challenge is localized in the mesh area. Systemic antibiotic treatment is often not effective in treating any ensuing infection.

What is needed then is an antimicrobial coating for surgical mesh, and a method of use, that provides adequate localized protection against pathogens that may cause infections. What is further needed is such a coated mesh that is non-toxic.

### SUMMARY OF THE INVENTION

Soft tissue repair mesh in current use comprises both synthetic and biological scaffolding, both allograft and xenograft material. Infection and foreign body response are issues that affect performance of these implant materials. The current invention discloses the attachment of various antimicrobial agents to both synthetic and biological meshes in such a way as to provide effective antimicrobial action that is often needed owing to large localized bacterial challenges resulting from large abdominal openings necessary for hernia repair, particularly ventral hernias. The antimicrobial agent must be bound to the mesh in an amount and in such a way as to allow the mesh to be inserted into the surgical field such that antibacterial action can take place both from leached (free) and bound antimicrobial molecules. The “microscopic” area of the mesh must be adequate to allow surface adsorption of ample antimicrobial agent molecules to counter the magnitude of the bacterial challenge, either through leaching, bound state action (i.e., covalent bonding), or both.

There are many know antimicrobial agents in the art that might be useful as coatings for implant mesh. Among them are lysostaphin, triclosan, ethanol, LL-37 peptide, various human defensins, and combinations of these and other antibiotics. The ideal requirements are that the agents should be non toxic and be readily attached to the mesh in such quantities as to be effective against large bacterial challenges, 10E8 CFUs/ml for example.

### BRIEF DESCRIPTION OF THE DRAWINGS

FIG. 1 is a graph illustrating the binding yield of lysostaphin on synthetic mesh;

FIG. 2 is a graph illustrating binding yield for lysostaphin on an allograft mesh material;

FIG. 3 is a graph illustrating the binding of lysostaphin to a xenograft mesh material;

FIG. 4 is a graph illustrating bind yield for triclosan on the synthetic mesh; and

FIG. 5 is a graph illustrating the leaching of enzyme from the meshes as a function of time.

### DETAILED DESCRIPTION OF THE PREFERRED EMBODIMENTS

The present invention relates to methods and materials for treating a infections in a mammal, particularly where the infections are located at the site of tissue repair and accessed through a large opening in the body. An implant that has antimicrobial properties is used in the repairing the tissue. The implant may be a synthetic material (e.g., polypropylene or polyester) or a biological material (allograft or xenograft). The implant materials are incubated in antimicrobial solutions. When the antimicrobial solution contains lysostaphin, the implant material is preferably incubated at a temperature of between 0 and 40 degrees centigrade and more preferably between 20 and 25 degrees centigrade. Additionally, the incubation period is preferably between 0.1 and 18 hours. When the implant materials are incubated in triclosan, the temperature is between 30 and 70 degrees centigrade and more preferably about 50 degrees centigrade. The lysostaphin solutions used for incubation are a buffered saline solution that has a pH between about 5 and about 9, and more preferably between about 7 and about 7.8. The triclosan solutions is water with polyethelene glycol (PEG) and the PEG has a molecular weight of between 300 and 10,000 gm/mole and preferably about 2000 gm/mole. The antimicrobial activity is due to the direct contact of the antimicrobial molecules on the implant with the microbes and also due to leaching of the antimicrobial molecules from the implant.

The following examples are indicative of the preferred embodiments of the method of utilizing and applying this invention:

### Example 1

### Binding of Lysostaphin to a Synthetic Mesh Material

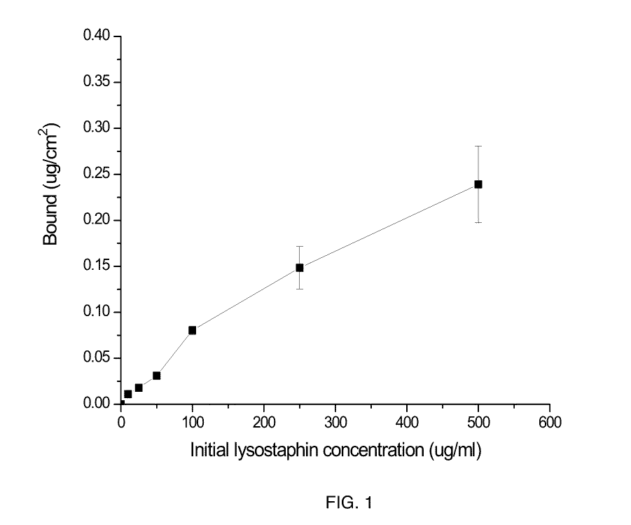

A synthetic mesh, Ultrapro, a lightweight polypropylene mesh manufactured by Ethicon, Inc., was cut into 1×1 cm pieces in a laminar flow hood under sterile conditions prior to adsorption of an enzyme. Initial enzyme concentrations of 10, 25, 50, 100, 250, and 500 micrograms/ml in PBS buffer were prepared from a 1 mg/ml stock solution of Alexa Fluor 594-labeled lysostaphin. The initial fluorescence intensities of the enzyme sample solutions (1 mL) were measured in a 12 well plate using a microplate reader (Ex 594 nm; Em 625 nm). The samples were then added to 25 ml sterile glass vials. Using a pair of sterile tweezers, mesh pieces were gently placed into each of the vials containing the enzyme solutions and incubated overnight at room temperature with gentle shaking (100 rpm). The enzyme solution over the mesh was then collected and stored for fluorescence measurements, and the mesh was gently washed 2 times with 1 ml of PBS buffer. The wash solution was also collected and used in determination of enzyme binding yield. To remove any loosely adsorbed enzyme, 1 ml of 0.1 (v/v %) Tween 20 solution (non ionic surfactant) was then added to each of the glass vials followed by incubation for 3 hours. This surfactant solution was also collected and used in the determination of the amount of desorbed enzyme, and the mesh samples were then washed with copious amount of PBS buffer. The concentration of unbound enzyme in each of the supernatants and wash solutions was determined from fluorescence measurements. Initial enzyme solutions with known concentrations were used as the standards. The concentration of the unbound/desorbed enzyme at each step was then calculated and subtracted from initial concentration of enzyme present in the initial solution. The difference in the concentrations corresponded to the bound enzyme concentration on the mesh, which is illustrated in FIG. 1. Adsorption experiments were reproduced in triplicate. The graph in FIG. 1 depicts the binding yield of lysostaphin on the synthetic mesh.

### Example 2

### Binding of Lysostaphin to an Allograft Mesh Material

One square centimeter samples of an allograft mesh material Alloderm, manufactured by LifeCell Corporation, were incubated in a lysostaphin solution as described below.

The allograft material was cut into 1×1 cm pieces in a laminar flow hood under sterile conditions prior to physical adsorption after the samples were soaked in PBS buffer for rehydration.

The samples pieces were then placed in a sterile 50 ml conical tube and incubated in 30 ml PBS buffer (10 mM phosphate; 140 mM NaCl, 3 mM KCl; pH-7.4) at room temperature (RT) for 30 minutes. As per manufacturer's instructions (Calbiochem—Cat#524650) dissolving one PBS tablet in 1 liter of deionized Water yields 140 mM NaCl, 10 mM phosphate buffer, and 3 mM KCl, pH 7.4.

The solution was then discarded and the samples were then gently flushed several times with PBS buffer using a 5 ml pipette.

The samples were incubated in 30 ml of PBS buffer at room temperature for 30 minutes and the flushing step was repeated.

The lysostaphin (Sigma Aldrich—L7386; lyophilized powder—5 mg, Protein ˜50-70%; remaining NaCl) was re-suspended in 1 ml of sterile PBS.

Initial lysostaphin concentrations of 25, 50, 100, 250, and 500 micrograms/ml PBS buffer were prepared from a 1 mg/ml stock solution of Lysostaphin.

One ml of the protein samples was then added to sterile 25 ml glass vials in a laminar flow hood under sterile conditions.

The allograft mesh material samples were gently placed into each of the vials containing the protein solutions and incubated overnight at room temperature (preferably in an incubator/shaker).

The sample solution was removed after overnight incubation and then gently flushed several times with PBS buffer using a 1 ml pipette.

The samples were then stored prior to use at 4° C. in 1 ml of PBS buffer.

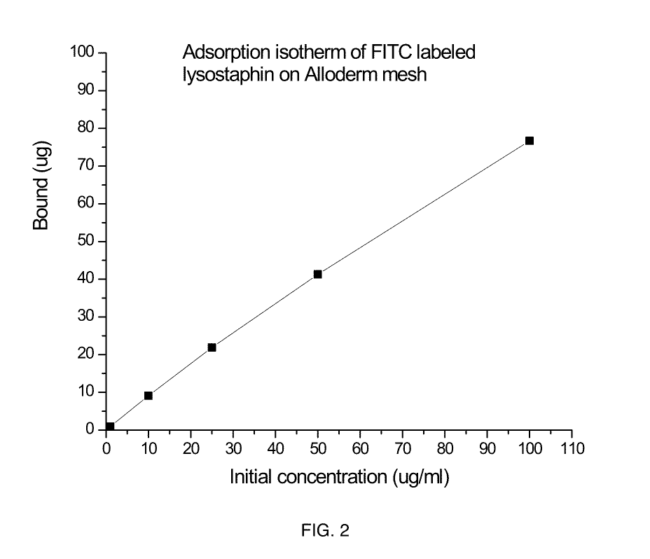

Binding yield was calculated based on the amount of fluorescently labeled enzyme adsorbed on mesh and corresponded to the difference in fluorescence intensities of initial enzyme and the supernatant solutions as described above for Example 1. FIG. 2 shows the binding yield for Alexa Fluor 594-labeled lysostaphin on a 1×1 cm allograft mesh material.

### Example 3

### Binding of Lysostaphin to a Xenograft Mesh Material

A xenograft mesh, Strattice manufactured by Life Cell Corporation, was soaked in sterile PBS buffer for 2 minutes according to manufacturer's instructions.

Buffer preparation: (10 mM phosphate; 140 mM NaCl, 3 mM KCl; pH-7.4) at Room Temperature for 5 min. As per manufacturer's instructions (Calbiochem—Cat# 524650). one PBS tablet was dissolved in 1 liter of deionized water to yield 140 mM NaCl, 10 mM phosphate buffer, and 3 mM KCl, pH 7.4.

The mesh was cut into 2×2 cm pieces in a laminar flow hood under sterile conditions, with the average weight of a 2×2 cm mesh being 1.25 g.

The mesh pieces were then placed in a 60 ml sterile wide mouth glass jar.

Lysostaphin solutions with concentrations of 1 and 100 micrograms/ml were prepared in PBS buffer from a 1 mg/ml stock solution of lysostaphin.

Two (2) ml of lysostaphin solution with concentrations of 1 and 100 micrograms/ml was then added to each of the glass jars containing the mesh samples in a laminar flow hood under sterile conditions and incubated for one hour at room temperature (preferably in an incubator/shaker).

The lysostaphin solution was discarded and the samples were washed three times with PBS buffer using a 5 ml pipette with 40 ml of buffer.

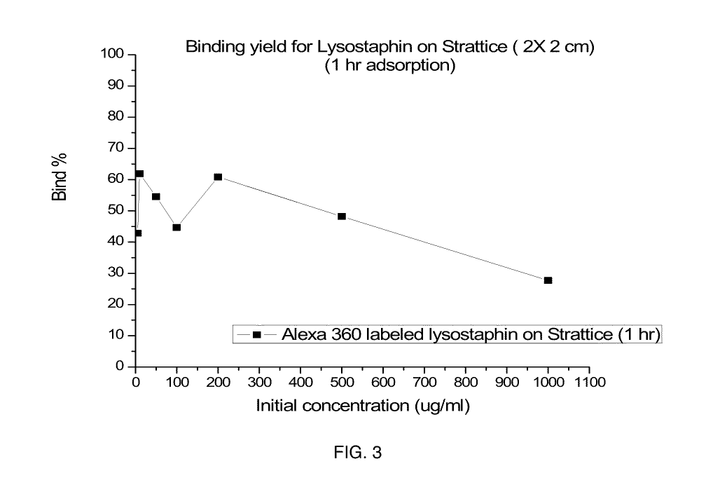

The binding yield of the lystostaphin xenograft is shown in FIG. 3.

### Example 4

### Binding of Triclosan to a Synthetic Mesh Material

Triclosan was placed into a vial and deionized water containing 5% PEG-2000 (w/v) was added to 1 obtain ml of solution.

The solution was heated at 50° C. for 8 minutes at 200 rpm, Triclosan dissolved completely to give a clear solution, and the initial stock solution of was found to be a 5% triclosan (wt/vol).

From this stock, solutions of various concentrations (wt/vol), 2%, 1%, 0.5%, 0.1%, and 0.01% were prepared and each sample was brought up to 1 ml using 5% PEG-deionized water solution.

From these solutions, 200 μl aliquots were transferred to the wells along with a well containing plain PEG-water solutions and their absorbance was measured using a UV-Vis spectrophotometer at 280 nm.

The aliquots were transferred back to the corresponding 1 ml vials and six 1×1 cm strips of the mesh Ultrapro, a lightweight polypropylene mesh manufactured by Ethicon, Inc., were cut and placed in glass vials and the six 1 ml solutions of various concentrations of triclosan. The samples were allowed to soak in the solution for 18 hrs.

The solutions were then drained completely and placed in their respective 1 ml vials from which 200 μl aliquots were removed for final absorbance readings.

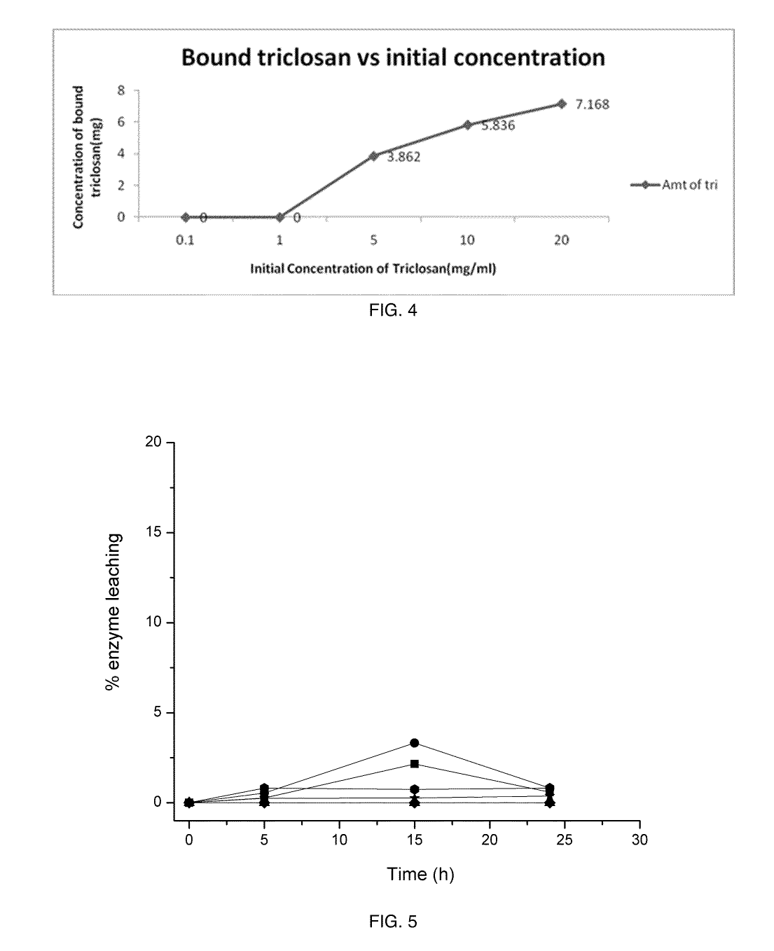

The bind yield for triclosan on the synthetic mesh is shown in FIG. 4.

### In Vitro Kill Rate of Staphylococcus aureus by Antimicrobial Materials

Mesh samples prepared as described above were placed in a cell suspension of Staphylococcus aureus (S. aureus). The bacteria suspension was prepared by inoculating 10 ml of 30% (w/v) tryptic soy with 100 μl of S. aureus culture prepared according to ATCC instructions. The procedure used is the method employed by Schindler and Schuhardt (Schindler, 1964). The cells were incubated at 37° C. for 18-24 h under gentle shaking to grow to a mid-log phase. The cells were then pelleted at 3,000 g for 10 min and washed twice with 10 mM PBS. The cells were then re-suspended in PBS to prepare bacterial suspension with optical density (OD600) of 0.7 at 600 nm (1 cm light path). One (1) ml of such suspension containing 10E7 CFU (colony forming units) was added to 25 ml glass vials containing the mesh samples. The samples were incubated with the suspension at 37° C. under continuous shaking, and the rate of bacerial lysis was monitored for 5 hours by taking 0.2 ml aliquots and measuring the OD600 in a 96 well plate at different time intervals. The mesh samples were placed in individual vials containing the bacteria suspension, which were continuously shaken to maintain contact with the meshes.

### Colony Counting Antimicrobial Assay

The sample meshes were placed in sterile glass vials and challenged with an inoculum of 1 ml of S. aureus suspension in tryptic soy broth containing 10E7 CFU for 24 hours at 37° C. After the incubation, the mesh pieces were retrieved and the broth supernatant was collected for subsequent analysis. Each mesh piece was then washed once vigorously with 1 ml of PBS buffer using a pipette followed by vortex washing for 5 minutes and collected for subsequent colony counting analysis. Serial dilutions and spot plating were then performed. All counts were performed in triplicates and results were calculated as mean log reduction±SD.

### Kill Rate of Lysostaphin on Synthetic Mesh

The table below shows the bacteria count rate after 5 hours of the bacteria solution after exposure to the synthetic mesh coated with various concentrations of lysostaphin.

| | Initial | Bound | | |
|---|---|---|---|---|
| | coating | lysostaphin | | |
| | concentration | concentration | CFU/cm² | |
| Sample | (μg/ml) | (μg/cm²) | (n = 3) | (±) SD |
| L - 10 | 10 | 0.0109 | 1.2 × 10⁵ | 7.8 × 10⁴ |
| L - 25 | 25 | 0.0179 | 9.4 × 10⁴ | 6.1 × 10⁴ |
| L - 50 | 50 | 0.0310 | 4.5 × 10⁴ | 3.7 × 10⁴ |
| L - 100 | 100 | 0.0804 | 3.7 × 10⁴ | 2.9 × 10⁴ |
| L - 250 | 250 | 0.1483 | 1.2 × 10⁴ | 1.4 × 10⁴ |
| L - 500 | 500 | 0.2391 | 5.3 × 10⁴ | 4.9 × 10⁴ |
| Uncoated | 0 | 0 | 1.5 × 10⁶ | 3.2 × 10⁴ |
| mesh | | | | |
| (control) | | | | |

### Kill Rate of Lysostaphin on Allograft Mesh

The 100 microgram/ml allograft mesh samples were inserted into a S. aureus solution prepared as described above and incubated for 24 hours. A kill rate of 99.2% was measured from the count.

### Kill Rate of Lysostaphin on Xenograft Mesh

The 100 microgram/ml xenograft mesh samples were inserted into a S. aureus solution prepared as described above and incubated for 24 hours. A kill rate of 97.8% was measured from the count.

### Kill Rate of Triclosan on Synthetic Mesh

The 20 mg/ml synthetic mesh samples were inserted into a S. aureus solution prepared as described above and incubated for 24 hours. A kill rate of 92.1% was measured from the count.

### Implantation into Rats

Three 3×3 cm samples of the above described lysostaphin coated meshes (100 micrograms/ml), of the allograft, xenograft and synthetic materials, were implanted under the skin onto the abdominal wall of 200 gram rats. S. aureus challenge of 10E8 CCU/ml was injected onto the mesh and the skin closed. Controls consisted of implanted mesh without the antimicrobial coatings. There were seven rats in each arm of the study. All seven rats in the control arm of the biological meshes (no antimicrobial coating) died prior to the 28 day end point. All of the biological mesh rats with lysostaphin were alive and showed no ill effects of the large bacterial challenge. Bacterial counts at the 28 day end points showed similar results to the in vitro studies. After 60 days the mesh in the controls (high bacterial challenge and no lysostaphin) had completely dissolved while the coated meshes were clearly intact and exhibited tensile strengths equivalent to the initial implant values.

### Toxicity

Lysostaphin toxicity studies conducted in rabbits showed no signs of toxicity below 15 mg/kg of body weight. Lysostaphin Treatment of Experimental Methicillin-Resistant Staphylococcus aureus Aortic Valve Endocarditis, Antimicrob Agents Chemother. 1998 June; 42(6): 1355-1360, American Society for Microbiology. This would equate to 3000 micrograms for the rats used in this study, which is well below the maximum bonded to any of the meshes in this study. The rats showed no signs of toxicity poisoning. For a human with a body weight of 80 kilograms the equivalent threshold for toxicity would be 1.2 grams which is far below the total weight that would be bonded to typical mesh sizes of 600 mm² and 5 mgs.

### Leaching

The samples were incubated with 1 mL of 2% (w/v) BSA solution in PBS buffer for 24 h at 37° C. Aliquots of 0.1 ml from each of the samples were taken at different time points (5, 15, and 24 h) and added to a 96 well plate. A series of lysostaphin standard solutions with known enzyme concentrations was also prepared from the same stock solution and added to each of the wells. A 100 μl cell suspension of S. aureus in PBS buffer with optical density (OD₆₀₀) of 0.3 at 600 nm (1 cm light path) was then added to each of the different wells and the rate of bacterial lysis was monitored continuously for 4 h at 37° C. The initial rate of the reaction for the different samples was calculated from linearized slopes and a standard curve was made by plotting the rate against the enzyme concentration. From the standard curve, the unknown lysostaphin concentration at different time intervals was calculated for each of the samples and the amount of enzyme leaching was plotted as a function of time as illustrated in FIG. 5.

We claim:

1. A method of treating infections in a mammal comprising:
providing a material having a therapeutic amount of antimicrobial properties; and
implanting the material in the mammal at an implant location to inhibit growth of pathogens at the implant location.

2. The method of claim 1, wherein the material is selected from a group of materials that includes a synthetic mesh, an allograft and a xenograft.

3. The method of claim 1, wherein the antimicrobial properties are a result of leached and attached antimicrobial molecules.

4. The method of claim 1, wherein the material is chosen from materials that include a polypropylene mesh and polyester mesh.

5. The method of claim 1, wherein the implant location is a wall includes the abdomen of the mammal.

6. The method of claim 3, wherein the antimicrobial molecules are one of the group including lysostaphin and triclosan.

7. A method of minimizing bacterial growth in a mammal undergoing soft tissue repair comprising:
providing a tissue repairing mesh;
coating the mesh with an antimicrobial agent;
implanting the mesh adjacent to the soft tissue to be repaired through a surgical opening; and
closing the surgical opening.

8. The method according to claim 7, wherein the mesh is a material selected from the group of materials including a biological material, an allograft and a xenograft.

9. The method according to claim 7, wherein the mesh is a synthetic material.

10. The method according to claim 7, wherein the mesh is hydrophilic.

11. The method according to claim 7, wherein the mesh is hydrophobic.

12. The method according to claim 7, wherein coating the mesh is by physical adsorption of the antimicrobial material by the mesh.

13. The method according to claim 7, wherein coating the mesh is by covalent bonding of the antimicrobial material onto the mesh.

14. The method according to claim 7, wherein the antimicrobial agent is pharmacologically effective against gram positive bacteria.

15. The method according to claim 14, wherein the antimicrobial agent is lysostaphin.

16. The method according to claim 7, wherein the antimicrobial agent is pharmacologically effective against gram positive and gram negative bacteria.

17. The method according to claim 16, wherein the antimicrobial agent is triclosan.

18. A method of coating a soft tissue repair mesh with an antimicrobial material comprising:
dissolving the antimicrobial material in a solvent to form an antimicrobial solution;
incubating the mesh in the solution at predetermined temperature for a predetermined time.

19. The method according to claim 19, where the antimicrobial material is lysostaphin.

20. The method according to claim 19, wherein the solvent is a buffered saline solution and the buffer has a PH between about 5 and 9.

21. The method according to claim 19, wherein the pH of the buffered saline solution is between about 7 and 7.8.

22. The method according to claim 19, wherein the predetermined temperature is between 0 and 40 degrees centigrade.

23. The method according to claim 19, wherein the predetermined temperature is between 20 and 25 degrees centigrade.

24. The method according to claim 19, wherein the predetermined time is between 0.1 and 18 hours.

25. The method according to claim 19, wherein the antimicrobial material is triclosan and wherein the solvent is water containing polyethelene glycol (PEG).

26. The method according to claim 19, the PEG has a molecular weight between 300 and 10,000 grams per mole.

27. The method according to claim 26, wherein the molecular weight is about 2000 grams per mole.

28. The method according to claim 25, wherein the PEG in the water is between 1 and 20 percent weight per unit volume.

29. The method according to claim 28, wherein the PEG in the water is about 5 percent weight per unit volume.

30. The method according to claim 19, wherein the predetermined temperature is between 30 and 70 degrees centigrade.

31. The method according to claim 30, wherein the predetermined temperature is about 50 degrees centigrade.
